(19) United States
(12) Patent Application Publication
(10) Pub. No.: US 2005/0042178 A1
(43) Pub. Date: Feb. 24, 2005

(54) Microparticles containing the CGRP-antagonist 1-[N2-[3,5-dibrom-N-[[4-(3,4-dihydro-2(1H)-oxoquinazoline-3-yl)-1-piperidinyl]carbonyl]-D-tyrosyl]-L-lysyl]-4-(4-pyridinyl)-piperazine, process for preparing and the use thereof as inhalation powder

(75) Inventors: Michael Trunk, Ingelheim (DE); Claudius Weiler, Ingelheim-Grosswinternheim (DE)

Correspondence Address: MICHAEL P. MORRIS;BOEHRINGER INGELHEIM CORPORATION, 900 RIDGEBURY ROAD, P. O. BOX 368, RIDGEFIELD, CT, 06877-0368, US

(73) Assignee: Boehringer Ingelheim International GmbH, Ingelheim (DE)

(21) Appl. No.: 10/901,791

(22) Filed: Jul. 29, 2004

Related U.S. Application Data
(60) Provisional application No. 60/503,116, filed on Sep. 15, 2003.

(30) Foreign Application Priority Data
Aug. 18, 2003 (DE) 103 38 399

(51) Int. Cl. A61K 38/04; A61L 9/04; A61K 9/14

(52) U.S. Cl. 424/46; 514/19; 514/252.17

(57) ABSTRACT

The invention relates to inhalable powders in the form of stable, spray-dried microparticles (embedding particles) for pulmonary or nasal inhalation, containing the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or a physiologically acceptable salt thereof and one or more excipients, processes for preparing such microparticles and the use thereof for preparing a powder inhalant for the treatment of headaches, migraine and cluster headache.

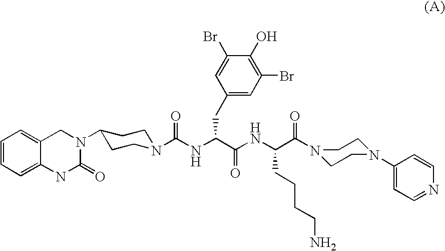

### FIELD OF THE INVENTION

The invention relates to inhalable powders in the form of stable, spray-dried microparticles (embedding particles) for pulmonary and nasal inhalation, containing the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or a physiologically acceptable salt thereof and one or more excipients, processes for preparing such microparticles and the use thereof for preparing a powder inhalant for the treatment of headaches, migraine and cluster headache.

### BACKGROUND TO THE INVENTION

The CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) is known from International Patent Application PCT/EP97/04862 (published as

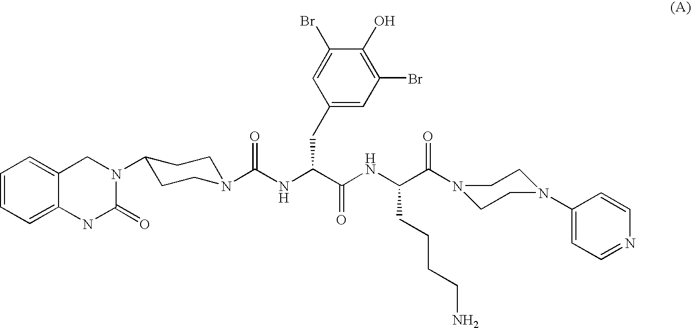

### PRIOR ART

The active substance base (A) is a highly effective CGRP antagonist for the acute and prophylactic treatment of headaches, particularly migraine and cluster headache, which cannot be administered orally using conventional formulations, as the substance has very limited oral bioavailability.

For treating attacks of migraine it is essential that an active substance is systemically available as quickly as possible. The treatment should be uncomplicated for the patient to administer and no other conditions which could affect bioavailability (e.g. the food effect) should restrict the use of the medicament for the patient.

Active substances which are intended to be systemically available are usually administered by oral route. If this route is unsuitable or undesirable on account of particular properties of the active substance or particular demands made of the application, other possible ways of administering substances systemically are known in the art. For example, inhalation, by means of which active substances may be administered systemically as well as topically, has been under discussion for some time. For substances which prove critical on account of their decomposition in solution or which have poor solubility per se, powder inhalation is an option. The absolute amount of the active substance which has to be administered per application makes particular demands of the formulation. On the other hand, the physical stability (e.g. aerodynamic particle size, dispersibility, physicochemical properties) of the active substance has proved to be a critical requirement for the development and production of an inhalable powder.

With formulations of the powder inhalant type, inhalable powders, which are packaged for example in suitable capsules (inhalettes), are delivered to the lungs by means of powder inhalers. Similarly, other systems in which the quantity of powder to be administered is pre-dosed (e.g. blisters), are also known as multidose powder systems. Alternatively, the medicament may also be inhaled by the use of suitable powdered inhalable aerosols which are suspended for example in HFA134a, HFA227 or mixtures thereof as propellant gas.

In powder inhalation, the microparticles of a pure active substance are administered through the airways onto the surface of the lungs, e.g. in the alveoli, by the inhalation process. These particles settle on the surface and can only be absorbed into the body after the dissolving process by active and passive transporting processes.

Inhalation systems are known in the literature in which the active substance is present in the form of solid particles either as a micronised suspension in a suitable solvent system as carrier or in the form of a dry powder.

Usually, powder inhalants, e.g. in the form of capsules for inhalation, are prepared on the basis of the general teaching as described in DE-A-179 22 07.

A critical factor in multi-substance systems of this kind is the uniform distribution of the pharmaceutical composition in the powder mixture.

Another important aspect of powder inhalants is that when the active substance is administered by inhalation only particles of a certain aerodynamic size reach the target organ, the lungs. The average size of these lung-bound particles (inhalable fraction) is in the region of a few microns, typically between 0.1 und 10 μm, preferably less than 6 μm. Such particles are usually produced by micronisation (air-jet grinding).

As a result, particles of this kind are often of complex composition as regards their crystal properties, because of this mechanical step. Similarly, the geometric shape of the particles of the starting material determines the morphological qualities of the micronised powder. For formulations of this kind it has proved essential to use a thermodynamically stable or the most stable form of the active substance in powdered preparations of this type. This is usually a crystalline form of the active substance.

It is known from the literature that particles in the submicron range can be produced by spray-drying. Usually, industrially suitable formulations which exhibit sufficient dispersibility in medical use (inhalation) may be prepared from spray-dried particles of this kind in accordance with the method cited above (DE-A-179 22 07) [Y.-F. Maa, P.-A. Ngyuyen, J. D. Andya, N. Dasovich, T. D. Sweeny, S. J. Shire, C. C. Hsu, Pharmaceutical Research, 15, No. 5 (1998), 768-775; M. T. Vidgrén, P. A. Vidgrén, T. P. Paronen, Int. J. Pharmaceutics, 35 (1987), 139-144; R. W. Niven, F. D. Lott, A. Y. Ip, J. M. Cribbs, Pharmaceutical Research, 11, No. 8 (1994), 1101-1109].

The spray-drying of pure active substances for the purposes of inhalation (powder inhalation) is also described in the prior art [e.g.: EP 0 072 046 A1 ; WO 2000 000176 A1; U.S. Pat. No. 6,019,968; A. Chawla, K. M. G. Taylor, J. M. Newton, M. C. R. Johnson, Int. J. Pharm, 108 (3), (1994), 233-240].

In addition to these examples there are other manufacturing techniques introduced particularly by pharmaceutical companies, based on spray drying methods, which prescribe special formulations for powder inhalants. Examples of these include: powder preparations consisting of co-spray-dried β-galactosidase with trehalose [J. Broadhead, S. K. Edmond Rouan, C. T. Rhodes, Pharm Acta Helvetiae, 70 (1995), 125-131], which may be mixed, for example, with other physiologically acceptable excipients; powder preparations consisting of a spray micronisate which consists by co-spray-drying at least two active substances and one or more physiologically acceptable excipients [WO 01/13885]; powder preparations prepared from spray-dried rhDNase, optionally co-spray-dried with salts, and either directly or in the form of a mixture with a physiologically acceptable excipient e.g. lactose, mannitol or sodium chloride for use by inhalation [H. K. Chan, A. Clark, I Gonda, M. Mumenthaler, C. Hsu, Pharm Research, 14 (1997), 431-437]; spray-dried IGF1 preparations for use by inhalation [WO 9955362]; co-spray micronisates consisting of active substances and physiologically acceptable excipients [WO 9952506] for use by inhalation; powder preparations containing co-spray micronisates consisting of SLPI protein in physiologically acceptable carrier materials [WO 9917800]; co-spray-dried interferon with a carrier material [WO 9531479]; co-spray micronisates consisting of an active substance and cellulose derivatives [WO 9325198]; co-spray micronisates consisting of RhDNase and a physiologically acceptable excipient, e.g. lactose, the primary amorphous excipient being converted into crystalline α-lactose monohydrate by subsequent recrystallisation [H.-K. Chan, I. Gonda, J. Pharm. Sci., 87 (5), (1998) 647-654].

In addition, the prior art also discusses special solutions for formulating active substances for delayed release, the active substances being incorporated in excipient matrices by methods based on the spray drying technique.

Apart from the solutions described above for formulating active substances in powder inhalants on the basis of the spray-drying technique it must be generally borne in mind that any change to the solid state of a pharmaceutical composition or to the active substance used which improves its physical and chemical stability and its technical properties gives a considerable advantage over less stable forms of the same pharmaceutical composition. Different physical/physicochemical properties may in some cases bring about improved pharmacological/pharmacokinetic properties of the pharmaceutical composition.

Statement of the Problem

The problem of the present invention is to provide a novel, stable formulation for the active substance base 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or a physiologically acceptable salt thereof using a carrier material. The formulation should preferably be of a composition such that the bitterness of the active substance is masked.

### DETAILED DESCRIPTION OF THE INVENTION

As already mentioned previously, a rapid onset of activity of the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or a physiologically acceptable salt thereof for the treatment of acute pain in migraine as well as a high plasma level within a very short time can best be achieved through the lungs as the site of absorption, as well as by intravenous administration.

In the production of an inhalable powder for pulmonary (or nasal) inhalation the active substance (A) or a physiologically acceptable salt thereof is incorporated in physically stable manner as a solid in a solid matrix of an excipient. By a suitable choice of excipients which are compatible with the active substance, the active substance can be incorporated in the solid matrix by the formulation method according to the invention so as to stabilise it with regard to the oxidative sensitivity of the active substance, for example, by the choice of a physically and chemically stable excipient. In addition, this embedding also provides a way of masking the bitterness of the active substance during its administration as an inhalant.

The particles according to the invention are characterised in that the physico-chemical properties are determined primarily by the physico-chemical properties of the embedding material. In particular, it has proved advantageous here that the bitter taste of the active substance is minimised or masked during inhalation of the inhalable powder according to the invention compared with inhalation of the pure active substance.

The invention also comprises corresponding methods of producing such particles. These powders may be used both directly as powder inhalants (multidose systems, pre-metered multidose systems and single-dose systems) and also as components which are mixed with other (e.g. coarse-grained) excipient.

Surprisingly, it has been found that the active substance 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or a physiologically acceptable salt thereof can be morphologically changed by co-spray-drying with various excipients such that a powder of this kind can be placed directly in a primary packaging means without any further manufacturing steps, and in particular without the need to mix it with a coarser carrier material (excipient), and can be delivered (inhaled) therefrom by means of a powder inhaler. At the same time it is found that the micronisates thus produced are largely unaffected by moisture and hence there is no longer any need to take account of any correction factors arising from the hygroscopic nature of the pure active substance, when producing a medicament from embedded particles of this kind.

To produce these particles the manufacturing process can be controlled so that the particles are obtained in a suitable particle size, usually between 0.1 and 10 μm, and these particles have surface properties such that they are easily vortexed or dispersed.

It has been found that the particle morphology including the particle size can be deliberately controlled by the choice of process parameters and production parameters.

All in all, a formulation based on this manufacturing process enables the active substance (A) or a physiologically acceptable salt thereof to be administered to the patient by inhalation in a therapeutically appropriate dose.

Particles produced by the process according to the invention are characterised by high physical stability. In particular, they are suitable when used as a powder inhalant if a high fine content is produced on delivery, technically measured by cascade impactor measurement, for example (Andersen cascade impactor, according to USP 254 or Pharm. Eur. Suppl. 2002). Typically, the proportion of particles according to this method which are less than 5 μm in size (aerodynamically) is greater than 15%; in some cases, even, fine contents of more than 50% are achieved. In addition to this key parameter for inhalants the powder is characterised in that it can be packaged directly by the usual filling methods, it is not absolutely essential to mix it with a physiologically acceptable excipient, but this may be mentioned as another alternative feature of the present invention.

Powders produced in this way are characterised by the physicochemical parameters of particle size, measured for example by laser diffraction, and specific surface area, measured for example by multipoint B.E.T. measurement. For the characteristic Q_{(5.8)} the particle size of powders produced in this way is typically between 50% and 100% and for the parameter X₅₀ in the range from 1 μm to 10 μm, preferably from 1 μm to 6 μm. Particles produced by the above methods typically have values for the specific surface area of between 1 m²/g and 20 m²/g, ideally between 1 m²/g and 10 m²/g. In terms of geometry, depending on the excipient used, particles prepared by the processes described above have particle shapes which may be described, depending on the test conditions, between the extremes of “spherical shape”, “spherical shape with cavity, possibly with hole”, “spherical shape with inwardly shaped convexities ”, and “collapsed hollow body”. Under the scanning electron microscope the surface of such particles is substantially smooth or spherically nanostructure on the surface. When mannitol is used as excipient, for example, recrystallisation of this substance takes place spontaneously during the manufacturing process, so that the particle morphology will also change from a spherical to rhomboid shape.

In a first aspect the present invention thus relates to an inhalable powder for pulmonary or nasal inhalation, comprising as active substance the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or a physiologically acceptable salt thereof and one or more excipients in the form of spherically nanostructured microparticles, which are characterised in that
(a) the particles have a specific surface area of between 1 m²/g and 20 m²/g, preferably between 1 m²/g and 10 m²/g,
(b) the characteristic Q_{(5.8)} is between 50% and 100%
(c) the parameter X₅₀ in the range from 1 μm to 10 μm, preferably from 1 μm to 6 μm.

According to the invention in addition to the active substance base (A) the physiologically acceptable acid addition salts are used which are selected for example from among 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, sulphate, phosphate, hydrobromide, carbonate, methanesulphonate, p-toluenesulphonate, nitrate, citrate, malate, tartrate, lactate, succinate, gluconate, acetate, formate, propionate, capronate, oxalate, maleate, fumarate, mandelate and hydroxysuccinate, while the 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, the sulphate and the hydrobromide are particularly preferred and the 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride is most particularly preferred.

Suitable excipients according to the invention include for example carrier materials selected from among the physiologically inactive polysaccharides (such as maltodextrin, starch, cellulose, dextrane), polylactide/polyglycolide (such as Resomer®), disaccharides (for example trehalose, lactose, maltose, saccharose), monosaccharides (for example fructose, glucose), polyalcohols (for example mannitol, sorbitol), amino acids (for example arginine hydrochloride), chitosan and mixtures of these carrier materials, the excipients trehalose, lactose, polylactide/polyglycolide, saccharose, maltodextrin, mannitol and mixtures of these excipients being preferred.

It is also possible to use excipients which preferably influence the surface properties of the microparticles (for example flow agents or lubricants). Examples include magnesium stearate, calcium stearate, stearic acid, stearylalcohols, calcium behenate, calcium arachinate, hydrogenated vegetable oils such as for example hydrogenated castor oil or hydrogenated cottonseed oil, fatty acid esters, sodium stearyl fumarate, sodium dodecyl sulphate, magnesium dodecyl sulphate, phospholipids or mixtures of the above-mentioned excipients.

Similarly, combinations of one or more carrier materials with one or more excipients which affect the surface properties of the microparticles are also possible.

The process for manufacturing the microparticles or inhalable powders according to the invention is characterised in that a solution of the active substance (A) or a physiologically acceptable salt thereof is suitably dissolved with one or more excipients, sprayed and dried in a spraying tower. The particles or powder may be obtained by a suitable precipitation process (e.g. cyclone or fine particle filter). The microparticles thus produced are characterised by special values in terms of particle size, specific surface area and morphology.

It has proved suitable to dissolve the active substance with one or more excipients in water, an organic solvent or an organic-aqueous solvent mixture. Solvents which may be used according to the invention include, in addition to water, organic solvents with a boiling point between 40° C. and 130° C., preferably alcohols.

Particularly preferably ethanol, methanol, propanol, dichloromethane, water or a mixture of these solvents is used according to the invention.

On the one hand the total quantity of solids in solution in conjunction with the spray-drying method determines the formation of the solid particles in terms of their particle size and morphology (and hence, indirectly, their inhalability) and on the other hand the relative proportion of active substance to the excipient causes the active substance to be incorporated homogeneously in the excipient, irrespective of the nearness or remoteness of the active substance molecules, and the active substance is physically and chemically stabilised by this “structuring” of the excipient. Surprisingly, it is found that in this way physically stable microparticles can be produced which allow a high proportion of active substance. It is possible to achieve mass ratios of active substance to excipient of 1:10 to 100:1. Ratios of between 1:3 and 20:1 are preferred. However, it is also possible to have micronisates which contain only traces of the excipient, for example in the form of a flavour component or an excipient which preferably influences the surface properties of the microparticle (e.g. flow agent), (relative compositions of active substance:excipient in the ratio from 50:1 to 5000:1, preferably 100:1 to 1000:1).

The concentration of solids in the spray solution serves to make the process economical. However, limits are set on the concentration of active substance to be achieved, which are prescribed by the fact that the surface qualities of the particles, including the particle size, can be optimised by achieving a specific ratio between the droplet size and concentration of solids. Usually, a concentration of between 1 wt. % and 20 wt. %, preferably between 2 wt. % and 10 wt. %, most preferably between 3 wt. % and 8 wt. %. The droplet size which is to be selected during the process can be characterised by the parameter X₅₀, which is in the range from 1 μm to 20 μm, preferably from 1 μm to 8 μm and particularly preferably from 1 μm to 3 μm, and the characteristic Q_{(5.8)}, which is between 30% and 100% and preferably between 60% and 100%.

This is implemented technically by using a corresponding commercial nozzle, e.g. a two-substance nozzle, which has these characteristics depending on the atomising pressure applied and the resulting mass flow of the atomising gas and the spray rate (flow volume of “spray solution”). Besides the special conditions which have to be adhered to during the actual spraying process in order to generate droplets which are suitable for the drying process, it is found that the surface properties of the particles can also be positively/deliberately influenced by the choice of drying parameters. The critical characteristics which impinge on the drying step are the entry and exit temperature of the drying gas, as well as its flow volume depending on the geometry of the spray dryer. The exit temperature must be adapted to the process so that the powder has a low enough residual solvent content and hence adequate chemical and physical stability is achieved. This is ideally done by keeping the exit temperature in the region of the boiling temperature or slightly above. On the other hand, the inlet temperature of the drying gas should be selected so that in conjunction with the parameter of the flow volume of the drying gas and the spray rate the drying is gentle enough to form particles with suitable surface properties. Particles according to the invention may be obtained by this process by applying the following parameters:
entry temperature of the drying gas from 100° C. to 350° C., preferably from 120° C. to 250° C. and particularly preferably from 130° C. to 200° C.,
an exit temperature of the drying gas from 40° C. to 120° C. and
a flow volume of the drying gas from 15 Nm³/h to 1500 Nm³/h, preferably from 15 Nm³/h to 150 Nm³/h.

The spray process is additionally carried out with a flow volume of spray gas of from 1 Nm³/h to 15 Nm³/h, preferably from 3 Nm³/h to 15 Nm³/h.

In a second aspect the present invention thus relates to a process for preparing microparticles in the form of embedding particles, containing the active substance, the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or a physiologically acceptable salt thereof and one or more excipients, comprising the steps of
(a) dissolving the active substance in water, an organic solvent or an organic-aqueous solvent mixture to prepare a solution of the active substance with a concentration of active substance of between 1 wt. % and 20 wt. %, preferably between 2 wt. % and 10 wt. %, particularly preferably between 3 wt. % and 8 wt. %,
(b) adding one or more excipients in a ratio of active substance:excipient of from 1:10to 100:1, preferably from 1:3to 20:1,
(c) spraying the resulting solution in the usual way, so as to obtain a spray mist with a droplet size with
   (i) the characteristic Q_{(5.8)} between 50% and 100% and
   (ii) the parameter X₅₀ in the range from 1 to 20 μm, preferably from 1 μm to 8 μm, particularly preferably from 1 μm to 3 μm, and
(d) drying the resulting spray mist using a drying gas while applying the following parameters:
   (i) an entry temperature of the drying gas from 100° C. to 350° C., preferably from 120° C. to 250° C. and particularly preferably from 130° C. to 200° C. and
   (ii) an exit temperature of the drying gas from 40° C. to 120° C. and
(e) separating the dried particles of solid from the current of drying gas in the usual way.

According to the invention it is preferable to use a process comprising the steps of
(a) dissolving the active substance in water, an organic solvent or an organic-aqueous solvent mixture, to prepare a solution of the active substance with a concentration of active substance of between 1 wt. % and 20 wt. %, preferably between 2 wt. % and 10 wt. %, particularly preferably between 3 wt. % and 8 wt. %,
(b) adding one or more excipients in a ratio of active substance:excipient of from 1:10 to 100:1, preferably from 1:3 to 20:1,
(c) spraying the resulting solution in the usual way, so as to obtain a spray mist with a droplet size with
   (i) the characteristic Q_{(5.8)} between 50% and 100% and
   (ii) the parameter X₅₀ in the range from 1 to 20 μm, preferably from 1 μm to 8 μm, particularly preferably from 1 μm to 3 μm,
(d) drying the resulting spray mist using a drying gas while applying the following parameters:
   (i) an entry temperature of the drying gas from 100° C. to 350° C., preferably from 120° C. to 250° C. and particularly preferably from 130° C. to 200° C.,
   (ii) an exit temperature of the drying gas from 40° C. to 120° C.,
   (iii) a flow volume of the spray gas from 1 Nm³/h to 15 Nm³/h, preferably from 3 Nm³/h to 15 Nm³/h and
   (iv) a flow volume of the drying gas from 15 Nm³/h to 1500 Nm³/h, preferably from 15 Nm³/h to 150 Nm³/h, and
(e) separating the dried particles of solid from the current of drying gas in the usual way.

Suitable excipients which may be used in the process mentioned above include, for example, the carrier materials or those excipients which influence the surface qualities of the microparticles and which have already been mentioned in the first aspect of the invention.

In a third aspect the present invention thus relates to the use of the embedding particles, i.e. particles consisting of the active substance and one or more excipients, which may be obtained by the process described above, in order to prepare a powder inhalant.

Experimental Section

1) Methods of Measurement

a) Determining the Droplet Size by Laser Diffraction
Measuring method: In order to determine the droplet size the spray cone (spray) of the nozzle is analysed directly in the laser measuring zone with regard to the droplet size distribution. The median value X₅₀ refers to the droplet size below which 50% of the quantity of droplets fall. The Q_{(5.8)} value describes the percentage of droplets which are less than 5.8 μm in size. H₂O is used as the solution.
Measuring device: Laser diffraction spectrometer (HELOS), Messrs. Sympatec
Software: WINDOX Version 4
Dispersing unit: RODOS/dispersing pressure: 3 bar
Focal length: 100 mm [measuring range: 0.9 . . . 175 μm]
Evaluation method: Mie (V 4)

b) Determining the Particle Size by Laser Diffraction
Measuring method: In order to determine the particle size the powder is fed into a laser diffraction spectrometer using a dispersing unit. The median value X₅₀ refers to the particle size below which 50% of the quantity of particles fall. The Q_{(5.8)} value describes the percentage of particles which are less than 5.8 μm in size.
Measuring device: Laser diffraction spectrometer (HELOS), Messrs. Sympatec
Software: WINDOX Version 4
Dispersing unit: RODOS/dispersing pressure: 3 bar
Focal length: 50 mm [measuring range: 0.9 . . . 175 μm]
Evaluation method: HRLD (V 4)

### 2) EXAMPLES

### Example 1

### Spray Parameters Suitable for Co-Spray-Drying with Lactose from Ethanolic Active Substance Solution (Modified BÜCHI Spraying Tower):

| | |
|---|---|
| Concentration of solution | 6 g active substance (A) and 1 g lactose in |
| | 93 g of a 30% solution of ethanol in water |
| Droplet size Q_{(5.8)} | 60% |
| X₅₀ | 6.5 μm |
| Flow volume “spray rate” | 1.0 L/h |
| Spray pressure | 6.5 bar overpressure (N₂) |
| (Nozzle type) | (BÜCHI spray nozzle 0.7 mm, |
| | Art.-no. 04364) |
| Flow volume | 52 NL/min |
| “atomising pressure” (Nozzle | (BÜCHI spray nozzle 0.7 mm, |
| type) | |
| entry temperature | 150° C. |
| exit temperature | 100° C. |
| flow volume “drying gas” | 35 Nm³/h |
| cross section of drying tower | 100 mm |
| particle size Q_{(5.8)} | 98% |
| X₅₀ | 1.9 μm |

### Example 2

### Spray Parameters Suitable for Co-Spray-Drying with Mannitol from Ethanolic Active Substance Solution (Modified BÜCHI Spraying Tower):

| | |
|---|---|
| Concentration of solution | 3 g active substance (A) and 3 g mannitol |
| | in 94 g of a 35% solution of ethanol |
| | in water |
| Droplet size Q_{(5.8)} | 60% |
| X₅₀ | 6.5 μm |
| Flow volume “spray rate” | 1.0 L/h |
| Spray pressure | 6.5 bar overpressure (N₂) |
| (Nozzle type) | (BÜCHI spray nozzle 0.7 mm, |
| | Art.-no. 04364) |
| Flow volume | 52 NL/min |
| “atomising pressure” (nozzle | (BÜCHI spray nozzle 0.7 mm, |
| type) | Art.-no. 04364) |
| entry temperature | 150° C. |
| exit temperature | 100° C. |
| flow volume “drying gas” | 35 Nm³/h |
| cross section of drying tower | 100 mm |
| particle size Q_{(5.8)} | 97% |
| X₅₀ | 2.0 μm |

### Example 3

### Spray Parameters Suitable for Co-Spray-Drying with Trehalose from Aqueous Active Substance Solution (Modified BÜCHI Spraying Tower):

| | |
|---|---|
| Concentration of solution | 5 g (A) and 1 g trehalose in 95 g 0.06 M |
| | aqueous HCl solution |
| Droplet size Q_{(5.8)} | 62% |
| X₅₀ | 6.3 μm |
| Flow volume “spray rate” | 0.7 L/h |
| Spray pressure | 7.0 bar overpressure (N₂) |
| (Nozzle type) | (BÜCHI spray nozzle 0.7 mm, |
| | Art.-no. 04364) |
| Flow volume | 55 NL/min |
| “atomising pressure” (nozzle | (BÜCHI spray nozzle 0.7 mm, |
| type) | Art.-no. 04364) |
| entry temperature | 160° C. |
| exit temperature | 80° C. |
| Flow volume “drying gas” | 35 Nm³/h |
| cross section of drying tower | 100 mm |
| particle size Q_{(5.8)} | 94% |
| X₅₀ | 2.3 μm |

1. An inhalable powder for pulmonary or nasal administration by inhalation, comprising as active substance the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine 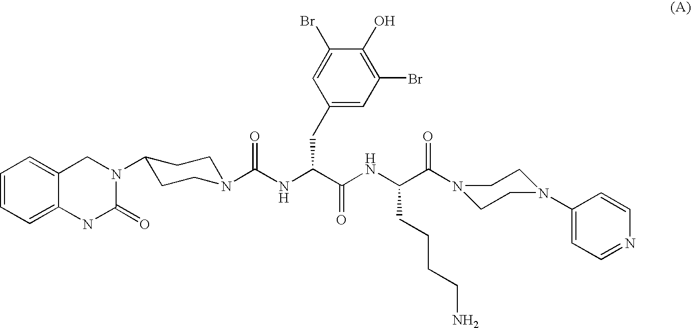
or a physiologically acceptable salt thereof and one or more excipients in the form of spherically nanostructured microparticles, characterised in that
(a) the particles have a specific surface area of between 1 m²/g and 20 m²/g, preferably between 1 m²/g and 10 m²/g,
(b) the characteristic Q_{(5.8)} is between 50% and 100% and
(c) the parameter X₅₀ is in the range from 1 μm to 10 μm, preferably from 1 μm to 6 μm.

2. The inhalable powder according to claim 1, characterised in that the physiologically acceptable salt is selected from the group consisting of 1-[N²-[3,5-dibromo-N-[[4-(3,4-dihydro-2(1 H)-oxoquinazolin-3-yl)-1-piperidinyl]carbonyl]-D-tyrosyl]-L-lysyl]-4-(4-pyridinyl-piperazine hydrochloride, sulphate, phosphate, hydrobromide, carbonate, methanesulphonate, p-toluenesulphonate, nitrate, citrate, malate, tartrate, lactate, succinate, gluconate, acetate, formate, propionate, capronate, oxalate, maleate, fumarate, mandelate and hydroxysuccinate.

3. The inhalable powder according to claim 1, characterised in that the physiologically acceptable salt is selected from the group consisting of 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride, sulphate and hydrobromide.

4. The inhalable powder according to claim 1, characterised in that the physiologically acceptable salt das 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine hydrochloride.

5. The inhalable powder according to claim 1, characterised in that the excipient(s) is or are selected from among inactive polysaccharides, polylactide/polyglycolide, disaccharides, monosaccharides, polyalcohols, amino acids, chitosan and mixtures of these excipients, the mass ratio of active substance:excipient being 1:10 to 100:1, preferably 1:3 to 20:1.

6. The inhalable powder according to claim 1, characterised in that the excipient(s) is or are selected from among maltodextrin, starch, cellulose, dextrans, Resomer®, trehalose, lactose, maltose, saccharose, fructose, glucose, mannitol, sorbitol, arginine hydrochloride, chitosan and mixtures of these excipients, the mass ratio of active substance:excipient being 1:10 to 100:1, preferably 1:3 to 20:1.

7. The inhalable powder according to claim 1, characterised in that the excipient(s) is or are selected from among trehalose, lactose, polylactide/polyglycolide, saccharose, maltodextrin, dextrans and mannitol, the mass ratio of active substance:excipient being 1:10 to 100:1, preferably 1:3 to 20:1.

8. The inhalable powder according to claim 1, characterised in that the excipient(s) is or are selected from among magnesium stearate, calcium stearate, stearic acid, stearylalcohols, calcium behenate, calcium arachinate, hydrogenated castor oil, hydrogenated cottonseed oil, fatty acid esters, sodium stearyl fumarate, sodium dodecyl sulphate, magnesium dodecyl sulphate, phospholipids and mixtures of these excipients, the mass ratio of active substance:excipient being 50:1 to 5000:1, preferably 100:1 to 1000:1.

9. A process for preparing microparticles in the form of embedding particles, containing as active substance the CGRP antagonist 1-[N²-[3,5-dibromo-N-[[4-(3,4-dihydro-2(1H)-oxoquinazolin-3-yl)-1-piperidinyl]carbonyl]-D-tyrosyl]-L-lysyl]-4-(4-pyridinyl)-piperazine (A) or a physiologically acceptable salt thereof and one or more excipients, comprising the steps of
(a) dissolving the active substance in water, an organic solvent or an organic-aqueous solvent mixture to prepare a solution of the active substance with a concentration of active substance of between 1 wt. % and 20 wt. %, preferably between 2 wt. % and 10 wt. %, particularly preferably between 3 wt. % and 8 wt. %,
(b) adding one or more excipients in a ratio of active substance:excipient of from 1:10 to 100:1, preferably from 1:3 to 20:1,
(c) spraying the resulting solution in the usual way, so as to obtain a spray mist with a droplet size with
(i) the characteristic Q_{(5.8)} between 50% and 100% and
(ii) the parameter X₅₀ in the range from 1 μm to 20 μm, preferably from 1 μm to 8 μm, particularly preferably from 1 μm to 3 μm,
(d) drying the resulting spray mist using a drying gas while applying the following parameters:
(i) an entry temperature of the drying gas of from 100° C. to 350° C., preferably from 120° C. to 250° C. and particularly preferably from 130° C. to 200° C. and
(ii) an exit temperature of the drying gas of from 40° C. to 120° C. and
(e) separating the dried particles of solid from the current of drying gas in the usual way.

10. The process according to claim 9, comprising the steps of
(a) dissolving the active substance in water, an organic solvent or an organic-aqueous solvent mixture to prepare a solution of the active substance with a concentration of active substance of between 1 wt. % and 20 wt. %, preferably between 2 wt. % and 10 wt. %, particularly preferably between 3 wt. % and 8 wt. %,
(b) adding one or more excipients in a ratio of active substance:excipient of from 1:10 to 100:1, preferably from 1:3 to 20:1,
(c) spraying the resulting solution in the usual way, so as to obtain a spray mist with a droplet size with
(i) the characteristic Q_{(5.8)} between 50% and 100% and
(ii) the parameter X₅₀ in the range from 1 μm to 20 μm, preferably from 1 μm to 8 μm, particularly preferably from 1 μm to 3 μm,
(d) drying the resulting spray mist using a drying gas while applying the following parameters:
(ii) an entry temperature of the drying gas from 100° C. to 350° C., preferably from 120° C. to 250° C. and particularly preferably from 130° C. to 200° C.,
(ii) an exit temperature of the drying gas from 40° C. to 120° C.,
(iii) a flow volume of the spray gas from 1 Nm³/h to 15 Nm³/h and
(iv) a flow volume of the drying gas from 15 Nm³/h to 1500 Nm³/h, preferably from 15 Nm³/h to 150 Nm³/h, and
(e) separating the dried particles of solid from the current of drying gas in the usual way.

11. The process according to claim 9, characterised in that water or alcohol-water mixtures, preferably ethanol-water mixtures or water-dichloromethane mixtures are used as solvent.

12. Microparticles made by the process of claim 9, 10 or 11.
